(19) United States
(12) Patent Application Publication
(10) Pub. No.: US 2015/0073513 A1
(43) Pub. Date: Mar. 12, 2015

(54) SYSTEMS AND METHODS FOR FACILITATING OPTICAL PROCESSES IN A BIOLOGICAL TISSUE

(71) Applicant: THE GENERAL HOSPITAL CORPORATION, BOSTON, MA (US)

(72) Inventors: Robert W. Redmond, Lancaster, MA (US); Seok-Hyun Yun, Belmont, MA (US)

(21) Appl. No.: 14/239,697

(22) PCT Filed: Aug. 27, 2012
(86) PCT No.: PCT/US2012/052451

§ 371 Date: Nov. 17, 2014

Related U.S. Application Data
(60) Provisional application No. 61/529,570, filed on Aug. 31, 2011.
(60) Provisional application No. 61/561,191, filed on Nov. 17, 2011.

(51) Int. Cl. A61N 5/06; G02B 6/02

(52) U.S. Cl. CPC A61N 5/0601; G02B 6/02033; A61N 2005/0643; A61N 2005/063
USPC 607/88

(57) ABSTRACT

A system and method for establishing optical communication between the depths of the biological tissue, optionally exceeding 1 cm, and the ambient environment with the use of an optical waveguide device that includes biodegradable material. The waveguide device is configured to deliver light from the outside into the biological tissue and/or vice versa. The light delivered from the biological tissue is informative about the status of the tissue. A specific waveguide device includes a mesh of biodegradable optical waveguides, is configured for insertion into the tissue, and does not require to be removed from the tissue after the irradiation of the tissue has been accomplished.

### CROSS-REFERENCE TO RELATED APPLICATIONS

The present application claims priority from and benefit of U.S. Provisional Patent Applications Nos. 61/529,570 filed on Aug. 31, 2011 and 61/561,191 filed on Nov. 17, 2011, a disclosure of each of which is incorporated herein by reference in its entirety.

### TECHNICAL FIELD

The present invention relates generally to systems and methods of light delivery to biological tissue and, more particularly, to activation and/or assisting light-based diagnostic and therapeutic processes by delivering light into and from the depths of biological tissue with the use of a biodegradable waveguide network.

### BACKGROUND ART

The use of electromagnetic (EM) radiation and, in particular, light for light-tissue interaction is recognized. Optically-controlled methods of treating biological tissues such as photodynamic therapy (PDT), photo-thermal therapy, low-level laser therapy, and light-activated drug release, to name just few, continue to emerge. With respect to the repair of injured skin and subcutaneous biological structures, for example, the use of non-ablative collagen remodeling (a so-called NCR technique) has been described that requires delivery of light or other form of EM energy (such as that at radiofrequencies) to assist in curing and cross-linking collagens in the tissue. Given that the NCR procedure generally relies on optimal coordination of EM energy delivery and cooling of the surface of the skin, a common side-effect of the NCR is that it is difficult to limit the zone of thermal damage, accompanying the NCR, in subcutaneous tissues.

On the other hand, while efficient delivery of light to and from the tissue is very important in clinical applications, the direct irradiation of the subcutaneous regions with EM radiation is difficult as the biological tissue itself and the skin efficiently scatter and/or absorb light at visible and near-infrared (near IR) wavelengths of interest and limit the depth of light penetration. In particular, the typical 1/e penetration depths of light into the biological tissue are only on the order of a few hundred micrometers or, at most, on the order of a millimeter. The related art discussed, for example, the use of fiber-optic-based catheters or lens-based endoscopes for light delivery into a body, but delivery of light at depths required by light-driven applications such as photochemical tissue bonding (PTB) and PDT, for example, remains elusive. Moreover, conventionally used systems facilitating light delivery into the subcutaneous layer to depths of about a millimeter (for example those employing hollow or fiber-optic based array of optical waveguides that puncture the skin to target the regions of interest (ROI) not directly illuminated through the skin) are typically made of generally non-biocompatible materials such as metal, glass, or plastic and, therefore, have to be removed from the body soon after use.

There remains, therefore, a need for a system and method that facilitate light delivery into a biological tissue at depths, such as at least dermatological depths or deeper (in order to, for example, photo-activate light-matter interaction processes in the tissue) and that do not cause trauma associated with a post-irradiation removal of the light-delivery system from the tissue.

### SUMMARY OF THE INVENTION

Embodiments of the present invention provide a system of light delivery to and from a biological tissue. Such system generally includes a light-guiding layer containing or made of biodegradable materials and having an optical terminal and a light-guiding surface. The light-guiding layer is adapted to emit light through the side surface when this side surface is brought in contact with the biological tissue. In one implementation, the light-guiding layer includes a slab waveguide that optionally has throughout openings in it. In a related implementation, the light-guiding layer includes a flexible and/or malleable network of optical waveguides and, in a specific embodiment, a mesh of optical waveguides that may be interwoven with one another. The mesh openings may be irregularly-shaped and preferably have dimensions that are substantially equal to the optical penetration depth of the coupled light into the biological tissue. In a specific configuration, the mesh of optical waveguides includes a tubular mesh.

The biocompatible and/or biodegradable material used for fabrication of the light-guiding layer may include a polymer and, in a specific embodiment, at least one of polyethylene glycols (PEGs), poly-L-lactic acid (PLLA), poly-dl-lactide-co-glycolide (PLGA) block copolymer, silk, collagen, and silk collagen block copolymer.

In one embodiment, waveguide(s) of the light-guiding layer include means configured to facilitate the outcoupling of light from the light-guiding layer through a light-guiding surface of the layer. For example, such outcoupling means may include particles dispersed throughout the waveguides, which either scatter or absorb the light incident onto the particles and, in a specific case, generate luminescent or fluorescent light in response to such absorption.

The light delivery system optionally further includes a source of light adapted to couple light into the optical terminal and an optical system configured to couple light from such source of light into the optical terminal of the light-guiding layer. Moreover, the system may additionally include an optical detector that receives light emanated from the tissue through the light-guiding layer.

Embodiments of the invention also provide a system for light delivery, which includes a biodegradable mesh of optical waveguides having respectively corresponding light-guiding surfaces. Such mesh has an optical terminal, and at least one of the optical waveguides forming the mesh is adapted to radiate light guided by such waveguide through a corresponding light-guiding surface when this surface is brought in contact with the biological tissue. The waveguide mesh is configured to be disposable in a crevice of a biological tissue at a depth of at least 1 cm. In a specific embodiment, at least one of said optical waveguides includes at least one of polyethylene glycols (PEGs), poly-L-lactic acid (PLLA), poly-dl-lactide-co-glycolide (PLGA). An embodiment of the system for light delivery may optionally include an opto-electronic component such as a source of light that is adapted to couple light into an optical terminal of the waveguide mesh, and/or an optical detector that is adapted to receive light guided by at least one of the optical waveguides through the optical terminal.

The waveguide mesh is additionally adapted to collect light through at least one light-guiding surface and deliver the collected light from the biological tissue towards an optical detector disposed in optical communication with the optical terminal. The waveguides of the waveguide mesh may additionally contain particles that are dispersed through at least one of the optical waveguides and that either scatter light incident upon them or generate fluorescent and/or luminescent light in response to such incident light. In a specific embodiment, the waveguide mesh may be shaped as a tube.

Embodiments of the invention additionally provide a method for establishing optical communication between a source of light and a receptor of light. Such method includes the steps of (i) receiving light from the source of light at an input portion of a biodegradable light-guiding layer that has light-guiding surfaces and openings through the light-guiding layer and that has been placed in proximity with the biological tissue; and (ii) outcoupling the received light from the light-guiding layer towards the receptor of light through at least one of optical terminals of the light-guiding layer. In one embodiment, the source of light includes a light source located outside of the biological tissue (for example, a laser), and a receptor of light includes a region of interest insider the depth of the tissue. In another embodiment, the source of light includes a light-emitting region of interest in the tissue and/or associated with the tissue (for example, a photo-activated dye disposed at depths of about 1 cm and greater in the tissue), and the receptor of light is an optical detector outside of the tissue. In particular, receiving light includes receiving light from the source of light at an input of a light-guiding layer having a mesh of optical waveguides that contain at least one of polyethylene glycols (PEGs), poly-L-lactic acid (PLLA), poly-dl-lactide-co-glycolide (PLGA). In another specific embodiment, receiving light includes receiving light at an input of a light-guiding layer having particles dispersed in the body of the light-guiding layer, and outcoupling light includes outcoupling at least one of (i) light scattered at these particles upon propagation through the light-guiding layer and (ii) fluorescent light generated at these particles in response to irradiation with light propagating through the light-guiding layer.

### BRIEF DESCRIPTION OF THE DRAWINGS

The invention will be more fully understood by referring to the following Detailed Description of Specific Embodiments in conjunction with the Drawings, of which:

FIG. 1A is a schematic diagram of a conventional fiber-optic based system for subcutaneous light delivery into the biological tissue.

FIG. 1B is a diagram illustrating schematically contrasting the outcome of conventional light-delivery processes with the use of a slab waveguide or a network of waveguides aggregated to form a mesh of waveguides of the invention.

FIG. 2 is a schematic diagram of a waveguide network according to the invention.

FIG. 3 is another schematic diagram of another waveguide network according to the invention.

FIG. 4 is still another schematic diagram of an alternative waveguide network according to the invention.

FIG. 5 is a schematic diagram of a biodegradable fiber-optic element of the invention.

FIG. 6 is a cross-sectional view of the biodegradable fiber-optic element of FIG. 5 showing particles dispersed across the fiber-optic element.

FIG. 7 is a cross-sectional view of a fiber-optic element for use with an embodiment of the invention.

FIG. 8 presents two chemical formulae describing material platforms for fabrication of the embodiments of the invention.

FIGS. 9A and 9B shows two slab-waveguides for use with embodiments of the invention.

FIG. 10A is an image of light-guiding mesh fabricated according to an embodiment of the invention.

FIG. 10B is a schematic illustration of biological tissue components to which irradiating light is delivered with and without an embodiment of the invention.

FIG. 10C presents images showing the depths of penetration, into the tissue, of light delivered with and without an embodiment of the light-guiding mesh of the invention.

FIG. 10D presents two graphs illustrating irradiance decay curves respectively corresponding to the images of FIG. 10C.

FIGS. 11A, 11B, and 11C are schematic illustrations of optical systems used for coupling of light into an embodiment of the invention.

FIGS. 12A, 12B, 12C, and 12D are diagrams showing schemes of spatial cooperation of an embodiment of the invention and the biological tissue.

FIG. 13 is an additional illustration of spatial cooperation of an embodiment of the invention and the biological tissue.

FIG. 14 is an illustration depicting an embodiment of the invention.

FIG. 15 is an illustration depicting the use of the embodiment of FIG. 14.

### DETAILED DESCRIPTION

In accordance with embodiments of the present invention, methods and apparatus are disclosed for light delivery into the biological tissue at depths significantly exceeding (for example, by an order of magnitude) typical depths associated with skin layers, with the use of an implantable waveguide network made of biocompatible and/or biodegradable materials that is placed at an opening of the biological tissue such as a wound and that does not require removal from the tissue.

References throughout this specification to “one embodiment,” “an embodiment,” “a related embodiment,” or similar language mean that a particular feature, structure, or characteristic described in connection with the referred to “embodiment” is included in at least one embodiment of the present invention. Thus, appearances of the phrases “in one embodiment,” “in an embodiment,” and similar language throughout this specification may, but do not necessarily, all refer to the same embodiment. It is to be understood that no portion of disclosure, taken on its own and/or in reference to a figure, is intended to provide a complete description of all features of the invention.

In addition, in drawings, with reference to which the following disclosure may describe features of the invention, like numbers represent the same or similar elements wherever possible. In the drawings, the depicted structural elements are generally not to scale, and certain components are enlarged relative to the other components for purposes of emphasis and understanding. It is to be understood that no single drawing is intended to support a complete description of all features of the invention. In other words, a given drawing is generally descriptive of only some, and not all, features of the invention. A given drawing and an associated portion of the disclosure containing a description referencing such drawing do not, generally, contain all elements of a particular view or all features that can be presented is this view in order to simplify the given drawing and the discussion, and to direct the discussion to particular elements that are featured in this drawing.

A skilled artisan will recognize that the invention may possibly be practiced without one or more of the specific features, elements, components, structures, details, or characteristics, or with the use of other methods, components, materials, and so forth. Therefore, although a particular detail of an embodiment of the invention may not be necessarily shown in each and every drawing describing such embodiment, the presence of this detail in the drawing may be implied unless the context of the description requires otherwise. In other instances, well known structures, details, materials, or operations may be not shown in a given drawing or described in detail to avoid obscuring aspects of an embodiment of the invention that are being discussed. Furthermore, the described features, structures, or characteristics of the invention may be combined in any suitable manner in one or more embodiments.

Moreover, if the schematic flow chart diagram is included, it is generally set forth as a logical flow-chart diagram. As such, the depicted order and labeled steps of the logical flow are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow-chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Without loss of generality, the order in which processing steps or particular methods occur may or may not strictly adhere to the order of the corresponding steps shown.

The invention as recited in claims appended to this disclosure is intended to be assessed in light of the disclosure as a whole.

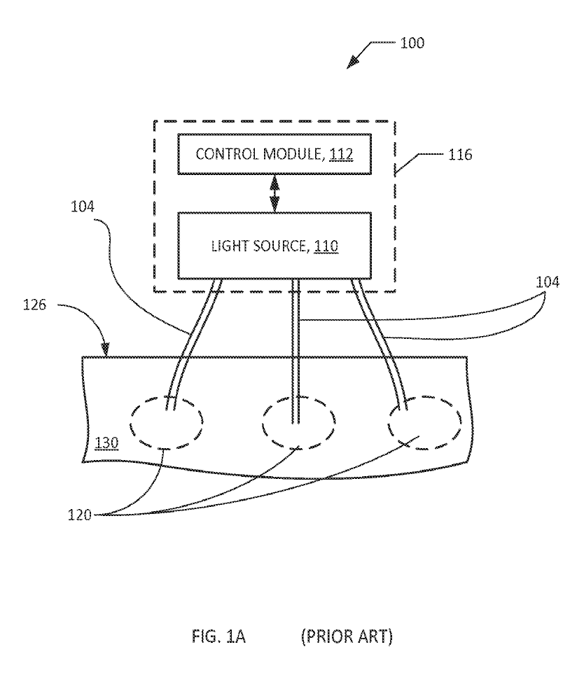

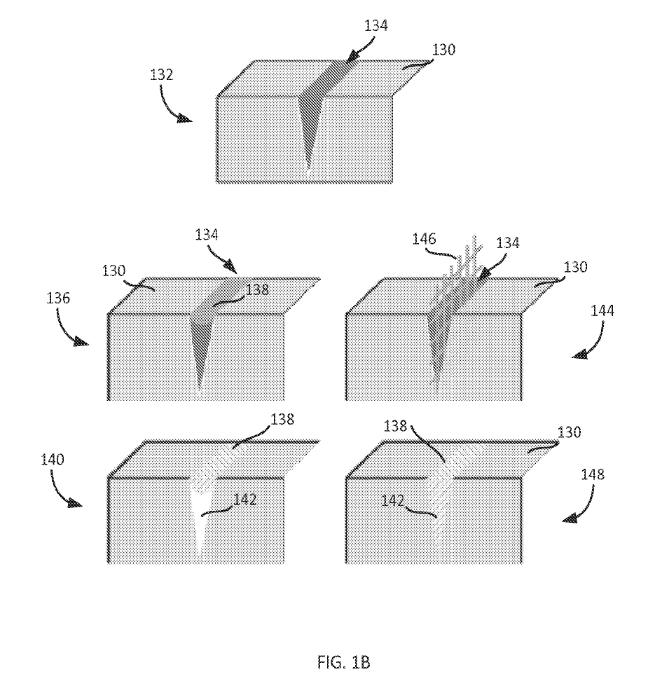

Conventional systems of light delivery, used to irradiate a target tissue located at depths on the order of a few millimeters in the body, have employed light-guide based devices (such as those employing fiber optic) that facilitate the delivery of photons to the subcutaneous target tissue. FIG. 1A is a schematic diagram of a conventional system 100 including an array of fiber-optic elements 104 (optionally structurally supported for higher rigidity, not shown), delivering the light from a light source 110 controlled by a control module 112 and optionally enclosed in a housing 116 to subcutaneous tissue regions of interest (ROIs) 120. The ROIs 120 are typically located at depths of about a millimeter or at comparable depths with respect to an upper surface 126 of tissue 130. While facilitating the desired delivery of light to the ROIs, these systems 100 employing fiber-optic elements 104 or other light-guide based devices require that the fiber-optic elements 104 be removed from the body after the goals of light delivery have been achieved. As previously explained, the removal of such fiber-optic elements 104 or other light-guide based devices presents a new trauma to the tissue 130, particularly about the upper surface 126 of the tissue 130, through which the fiber-optic elements 104 or other light-guide based devices extend.

Embodiments of the invention provide a system and method for facilitation of optical communication with biological tissues located not only subcutaneously but also at depths of at least 1 cm, both in vivo or ex vivo. According to the present invention and referring to FIG. 1B, a representation of a subject's tissue 130 is provided, as generally indicated at 132, as having a passage 134 formed in the tissue 130, such as that caused by trauma or surgical procedure. Should a clinician desire to support healing of the passage 134 using a traditional light-based therapy device the clinician can, as described above, utilize an invasive therapy device that must be removed from the tissue 130 once the therapy is complete and, thereby, introduce further trauma. Else, as illustrated in FIG. 1B, the clinician may use a conventional, non-invasive delivery that, as illustrated generally at 136, limits the depth of penetration of the supplied phototherapy to an upper level 138 of the tissue 130 and the passage 134. As generally indicated at 140, such non-invasive or superficial therapy deliveries, though beneficial, often result in the upper level 138 of the tissue 130 healing quickly, yet interior portions 142 of the tissue located thereunder, which did not benefit from receiving phototherapy healing at a different rate or in an otherwise less-desirable manner.

Instead, as illustrated generally at 144, a system in accordance with embodiments of the present invention may be utilized that includes a light-guiding layer 146 (such as a slab waveguide or a network of waveguides aggregated to form a mesh of waveguides, for example) having, as will be described, an optical terminal (not shown) and including biocompatible and biodegradable materials and cooperated with a biological tissue 130 such as to facilitate light guiding along a depth of the passage 134 in the tissue 130. In one embodiment, such biodegradable light-guiding layer 146 is implanted or embedded in the tissue 130 to deliver light into the tissue to initiate photophysical processes such as photoexcitation leading to generation of light and/or heat and/or photochemical processes and is gradually absorbed by or integrated into the tissue 130. Alternatively or in addition, the biodegradable light-guiding layer 146 element is adapted for light delivery from the depths to the outside of the tissue 130 for detecting changes in the condition of the tissue that represent themselves optically. An embodiment where the light-guiding layer 146 of the invention includes a plurality of individual waveguides (WGs), such waveguides may be generally configured as fiber optic (FO) elements, optical filaments, channel WGs, or a combination of the above, and may include, without limitations, a WG core and an optional WG cladding with predetermined index distribution profiles. Alternatively, an individual WG may contain a gradient-index (GRN) structure. Regardless of the specific structure employed, as generally indicated at 148, systems and methods in accordance with the present invention facilitate desirable therapeutic benefits at not only the upper level 138 of the tissue 130, but also interior portions 142 of the tissue located thereunder.

### Embodiments of WG-Network/Mesh

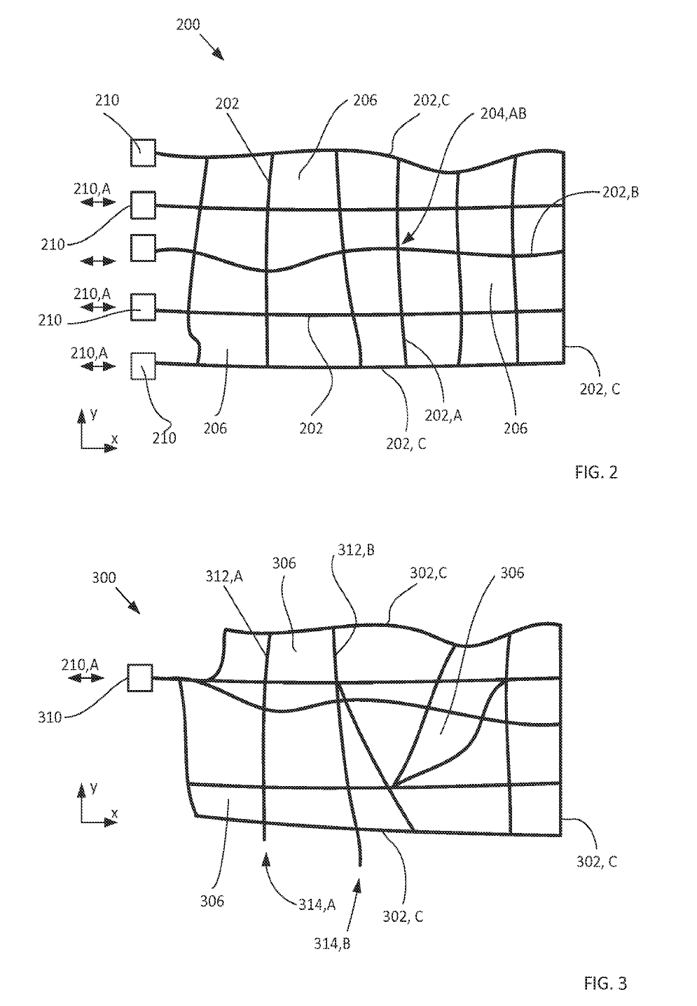

In accordance with an exemplary embodiment described with reference to FIG. 2, an optical waveguide element 200 is provided including a network of waveguides (WGs) 202 that are cooperated in a light-guiding layer, in xy-plane, and that are optically coupled with one another. The optical coupling between and among the WGs 202 is such that light guided by at least one of the WGs (for example, the WG 202,A) is at least partially redirected to another WG (for example, WG 202,B) at an intersection of the element 200 defined by the WGs in question (for example, at an intersection 204,AB). A WG intersection of the WG network may include a WG-splitter or, in the alternative, may be formed by waveguides configured in such proximity of one another that light coupling between the WGs occurs through evanescent field. In one embodiment, for example, the individual WGs 202 are interwoven (not shown) to define the mesh-network such as the network 200.

As shown in plan view of FIG. 2, the embodiment 200 contains a mesh of waveguides 202 with irregular mesh openings 206 defined by intersections of four WGs 202. Generally, however, a WG mesh (also interchangeably referred to as light-guiding mesh or LGM) of an embodiment of the invention may contain mesh opening of arbitrary form defined by intersections of at least two WGs. While individual WGs of the WG-network do not have to be aligned linearly and may contain bends, the cooperation of substantially straight WGs into the mesh may advantageous in at least one of fabrication of the WG mesh and its operation.

In further reference to FIG. 2, the WG-network layer 200 includes at least one optical terminal 210 configured to facilitate at least one of light coupling into and light outcoupling out of the WG network 200, as shown schematically by arrows 210A. The term optical terminal, as applied to an embodiment of the invention, conventionally refers to a portion of an embodiment that is configured to facilitate at least one of coupling and outcoupling of light into a light-guiding structure of the embodiment. In one implementation, such portion may include at least one of a waveguide (or optical fiber) facet and a waveguide (or optical fiber) surface, which may optionally be modified to increase the efficiency of light coupling/outcoupling (by adding, for example, a diffractive structure to the waveguide surface). In addition or alternatively, an optical terminal may include a waveguide taper, a coupling optic such as a lens, an optical beamsplitter, an optical filter (such as a thin-film interference filter, for example, a diffractive optical element, or a light polarizing component), and an and optical reflector, to name just a few. Other optical components can be used as required and as known in the art.

The WG-network layer additionally includes a perimeter waveguide 202,C that is adapted to establish optical communication among the facets of the WGs 202 defining the network 200. It is appreciated, however, that, generally, an embodiment of the WG network may have at least one individual WG that has a “loose” or free end optionally terminated with a facet through which the light guided in the embodiment of the WG network is outcoupled from such individual WG. Such facet is appropriately configured at a predetermined angle with respect to an optical axis of the individual WG, as known in the art.

An embodiment of the light-guiding layer of a WG network 300 that includes a WG mesh with a perimeter WG 302,C and that has irregular multisided mesh openings 306, a single input optical terminal 310, and two WGs 312,A and 312,B with corresponding free terminating facets 314,A and 314,B, is shown in plan view in FIG. 3. In reference to both FIG. 2 and FIG. 3, mesh openings 206 and 306 are preferably dimensioned to be on the order of or, optionally, smaller than the depth of penetration of light into the biological tissue, which is about 100 microns to a few millimeters. While a dimension of any given mesh opening can be made larger than a value of the light-penetration depth, the above-mentioned preferred dimensioning facilitates such irradiation of a tissue portion substantially co-extensive with a given mesh opening that does not leave a fraction of the tissue portion not illuminated. Mesh openings comparable in size to the width of individual WGs are also within the scope of the present embodiments.

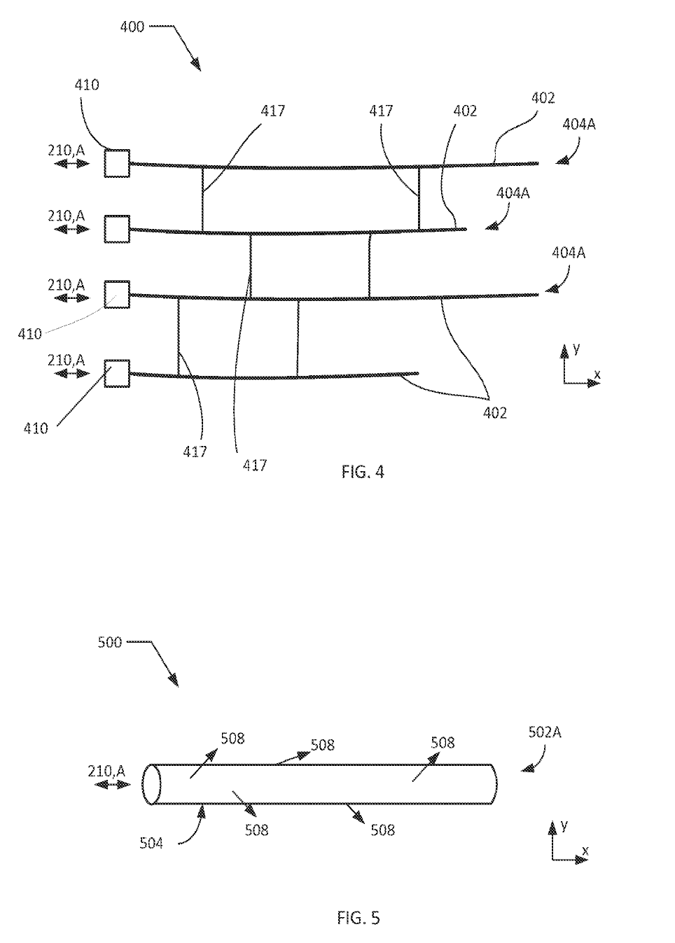

FIG. 4 illustrates a WG network 400 including an array of individual FO elements 402, generally having different lengths and terminating facets 404A and equipped with respectively-corresponding input optical terminals 410 adapted to couple light in and outcouple light out of the individual WGs 402. The WGs 402 are cooperated in a desired spatial relationship and interconnected with non-light-guiding supporting elements 417.

### Outcoupling Means/Particles

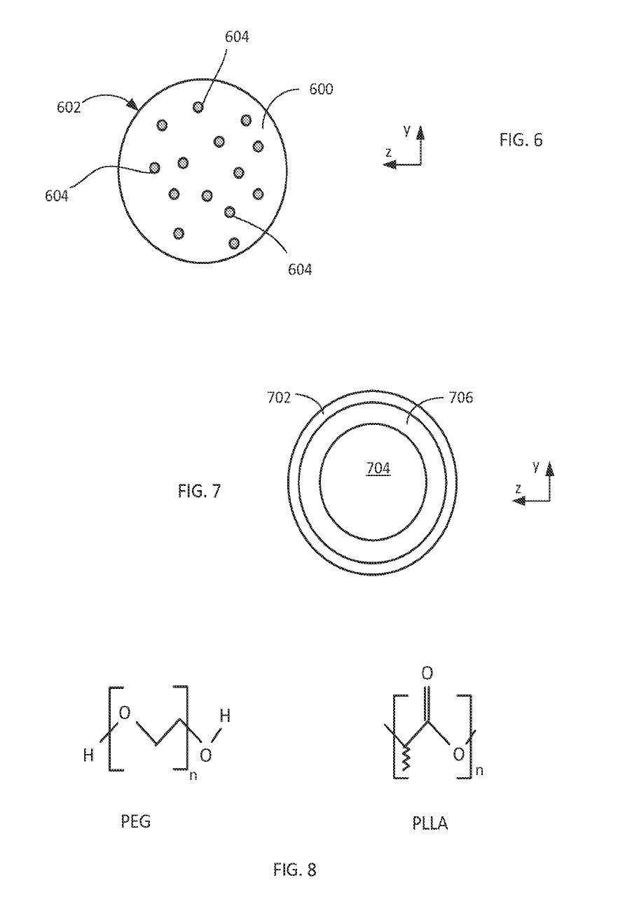

As shown in the example of a FO-element 500 with a end facet 502A of FIG. 5, in one embodiment at least one individual biodegradable WG defining WG-networks of the invention additionally contains a light-guiding surface 504 that is defined by a dielectric boundary formed by the embodiment of the WG and a light-outcoupling means configured to facilitate the outcoupling of guided light through light-guiding surface(s) 504 of the waveguide, as shown schematically by arrows 508. In one embodiment, a WG is placed in proximity with and/or in the biological tissue. Accordingly, light coupled into the light-guiding element 500 at a chosen optical terminal (as shown schematically by an arrow 210,A), is outcoupled along the length of the element 500 upon propagation in the WG. In one embodiment, such light-outcoupling means includes appropriately distributed (along the length of the WG in question) perturbations on the light-guiding surface(s) of the WG such as, for example, holes or openings or cavities in the WG (not shown). In another embodiment, such light-outcoupling means includes formatting the light-guiding surface(s) to include surface roughness of the predetermined value (such as, for example, a corrugation, not shown). In yet another implementation, the guided light is scattered and outcoupled outwardly upon interaction with micro- or nano-particles embedded into the light-guiding body of the WG element in a predetermined spatial fashion defining a desired profile of outcoupled light intensity along the length of the WG. A schematic of such biodegradable FO-element 600 having a light-guiding surface 602 and containing scattering particles 604, distributed across the body of the element 600, is shown in a cross-sectional view of FIG. 6.

The particles embedded into a light-guiding body of the WG may be adapted to emit fluorescence or luminescence in response to interaction with light guided by the WG, as a result of which the light outcoupled through the light-guiding surface(s) of the WG includes fluorescent or luminescent light. As known in the art, the spectrum of either of fluorescence and luminescence differs from that of excitation light. For example, the particles 604 may include biological cells engineered to emit fluorescent or luminescent light or to produce and release bio-chemicals to the surrounding tissue. In other related embodiments, the above-mentioned outcoupling of light may be facilitated with the use of outcoupling means including refractive-index match or index antiguiding mechanisms (for example, when the refractive index of the WG material is substantially equal to or lower than that of the surrounding tissue, respectively).

It is appreciated, therefore, that any of the above-discussed WG-network embodiments of FIGS. 2, 3, and 4 are preferably adapted to radiate guided light into the ambient medium (such as the biological tissue) not only from the terminating facets of the individual WGs but also at multiple points and along the length of the WG-network embodiment, in order to more uniformly irradiate the ambient medium across the area the size of which is comparable to that of the WG-network.

### Materials

According to embodiments of the invention, the WG-based systems of light delivery to and from the biological tissue are configured as systems that are implantable and/or embeddable into the tissue. The systems of the invention do not require removal from the tissue when the targeted light-matter interaction processes such as, for example, (i) irradiation of tissue with external light for the purposes of activating physical and chemical processes within the tissue or, alternatively, (ii) collecting light emitted from within the tissue in order to assess the physical, chemical, and/or biological condition of the tissue have been accomplished. The implantable configuration of the embodiments enables various types of light-matter interaction such as, for example, PTB within the depths of a biological tissue on the order of and exceeding 1 cm. Accordingly, embodiments of the invention include biocompatible and/or biodegradable materials, such as, for example, those including photo-crosslinkable hydrogels (and, in particular, mono and di-methyl-substituted polyethylene glycols or PEGs such as PEGMA and PEGDA); PLLA; PLGA block co-polymer, silk, and collagens, as well as hydrogels based on these polymers. Embodiments of the present invention lend themselves to continuous real-time monitoring and longitudinal studies that identify response(s) of tissues to natural processes and/or treatments designed to evoke a therapeutic effect.

Biodegradation typically requires the presence of at least one of water, oxygen, and enzyme and in some cases may be accelerated with light irradiation, thereby ensuring that a biodegradable implant or insert can be removed on demand. The biodegradation time can be defined with several metrics known in the art such as swelling and loss of weight. In addition, the degradation can be defined in terms of changes in optical properties, such as scattering coefficient and transmission loss. The degradation time for a WG structure and function may range from about a hour to about a year, depending on the materials used and the structure of the WG. For example, a thin 50/50 PLGA fiber may lose its initial optical and structural properties within a day and is reabsorbed by the body in about a week. In contrast, a thick cross-linked PEG fiber may maintain its shape and function for several months. It is appreciated that biodegradation of the materials used in fabrication of embodiments of the invention affects the optical transparency and transmission characteristics of the light-guiding structures. At the same time, the change in optical characteristics of the envisioned waveguiding elements may precede the biodegradation of the WG material itself. For example, pristine PLGA may absorb water and become opaque. Accordingly, in one embodiment, in order to control the change of optical characteristics of the FO elements defining the WG-mesh of the invention, different materials are used to fabricate the core, the cladding, and the coating of an optical fiber.

For example, in reference to FIG. 7, the coating 702 can be made of a material, the degradability of which is slow to provide a long-lasting protective layer to the core 704 and the cladding 706 (against, for example, moisture-induced swelling). The core 704 and/or the classing 706 can be made of high-transparency materials with relatively short degradation time. In different embodiments, the time of optical of a WG element of the invention (related to change of transmission characteristics of the WG element) may range from several minutes to a year, depending on the application. For example, the use of WG materials having a short time of optical degradation may be acceptable in applications such as an acute therapeutic treatment, while the use of WG materials ensuring a long-term stability of optical characteristics (and, therefore, long times of optical degradation) would be more appropriate for “fractionated” treatment (when the treatment is provided in a multitude of doses delivered at pre-determined time intervals) or long-term monitoring of the tissue status. PLGA copolymers are available with various concentration ratios or viscosities. The value of viscosity of such a copolymer at glass transition temperature can be adjusted by choosing the length of the polymer. The rate of biodegradation can be adjusted by modifying the lactide-to-glycole ratio. For example, the 50/50 PLGA has a fast biodegradation time

FIG. 8 shows two examples of chemical structures related to PEG and PLLA as material platforms that may be used for the fabrication of biodegradable/biocompatible optical components according to the embodiments of the invention. PEG is a biocompatible polymer. Mono- and di-methyl-substituted forms of PEG (PEGMA and PEGDA) are photo-crosslinkable materials and, in one embodiment, WG-elements of the invention are fabricated with the use of PEGMA and/of PEGDA via photolithography. In one embodiment, appropriate blending of PEGMA with PEGDA or other related hydrogels is used to tune the refractive index of the resulting material to optimize waveguiding properties of the resulting WG elements. As a high-molecular weight polymer, pristine PLLA is mechanically stable at body temperature. The WG-network and other biodegradable optical components including PLLA or related polymers, that are embedded into the biological tissue, can be absorbed by the tissue within a relatively short time (on the order of several weeks).

### Fabrication

Fabrication of the light-guiding and other optical components of the invention may be accomplished in different ways. In one embodiment, for example, a flexible or malleable channel waveguide is fabricated by printing or stamping the WG-mesh from a layer of the PLLMA- or LEG-based material. Alternatively, lithographic techniques (applied to PEGDA) and solvent-casting (applied to PLLA) can be utilized. In another embodiment, two PEG-based formulations (with slightly different refractive index) are passed through a double-layered glass or plastic capillary, to respectively define the core and cladding structures of the FO-element, towards the exit orifice where the drawn/extruded structure is crosslinked (by photo-curing with laser light or thermo-curing). In a related embodiment, the drawing of the crosslinked material from the capillary may be optionally assisted with at least one of vacuum and hydrostatic pressure and microfluidic technologies. The resulting LGMs are then fabricated by weaving the WG-mesh from linear FO-element(s). In either embodiment, the light-scattering particles such as particles 604 of FIG. 6 can be embedded into the polymer material prior to fabrication of the light-guiding element.

Generalization to a WG layer

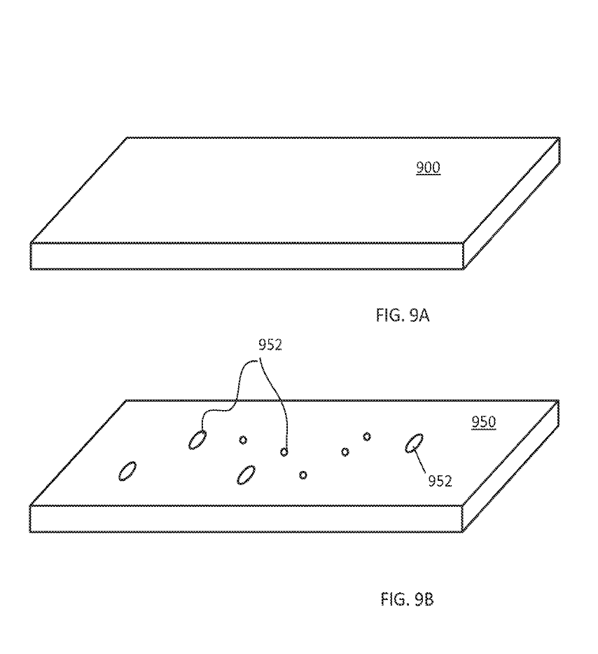

It is appreciated that the above-described WG-networks (such as LGMs of FIGS. 2, and 3, for example) are structures that include a generally quasi-continuous light-guiding layer defined by corresponding LGMs. Accordingly, a more general embodiment of an LGM of the invention includes a flexible or malleable slab WG, optionally having perforations/openings in it. FIGS. 9A and 9B are schematics of such slab WGs 900 and 950, shown spread in xy-plane in perspective views. Similarly to methods of fabrication disclosed above, the light-guiding layers 900, 950 can be fabricated by extrusion/drawing and/or casting or printing technologies (to ensure the formation of perforations 952 in the layer 950), and may include a multi-layered structure. In a fashion similar to that discussed in reference to FIG. 6, auxiliary light outcoupling means such as material particles can be dispersed throughout light-guiding bodies of the layer 900, 950. While not shown in FIGS. 9A and 9B, it is appreciated that at least one optical terminal such as terminal(s) 210 of FIG. 2, for example, can be cooperated with either of the layers 900, 950 to ensure the coupling of light into a corresponding layer.

### Light-Delivery System as a Whole, Including Sensor

The EM radiation from the source of light includes spectral components in the range from about 250 nm to about 2,000 nm, at power levels from about 100 microwatt to about 1 W. Embodiments of light-guiding layer are preferably configured to have low absorption and/or losses at wavelengths of interest, such that most of the EM radiation coupled into the light-guiding layer is emitted towards and into the tissue.

In a specific implementation, a portion of the biological tissue to which the biodegradable light-guiding layer delivers light from an outside source can be tagged or associated with at least one type of light-absorbing markers such as molecules of light-absorbing material disposed on some biological cells or, in addition or alternatively, in an extracellular matrix associated with the targeted portion of the tissue. For example, the light-absorbing material may include at least one of a fluorophore such as fluorescein, a photosensitizer such as Rose Bengal, a photo-cross-linking material such as riboflavin, a photodynamic agent such as photofrin, a photobiomodulator such as a calcium-releasing compound, photo-thermal nanoparticles such as gold nanoparticles, and photo-controllable ion channels administered to the tissue.

Alternatively or in addition, the same biodegradable light-guiding structure can be used to deliver light in the opposite direction, from the depths of the tissue, in which it is embedded, to an optical detector outside of the tissue. Such embodiment may be used to effectuate the registration of, for example, scattering processes, fluorescence, phosphorescence, chemiluminescence, and bioluminescence occurring within the tissue. Accordingly, in such case an embodiment of the invention may additionally include an optical detector (such as a photo-multiplier tube, a spectrograph, or a CCD) operably connected with an optical terminal and configured to receive light that has been coupled into the biodegradable light-guiding layer and delivered by this layer from inside the tissue. The spectral data contained in such light are representative of various characteristics describing the status and/or condition of the tissue such as, for example, pH, oxygenation, tissue viability, metabolic activity, presence or absence of a particular disease, composition, vascularity, perfusion, or other conditions of interest. Adapted as an optical sensor component, the operation of an embodiment of the light-guiding layer of the invention (such as, for example, the layer 200 of FIG. 2 or 400 of FIG. 4 or 900 or 950 of FIG. 9) can be supplemented with individual probes, materials, or markers such as molecules that are applied to or associated with the biological tissue in question. For example, prior to inserting or implanting a biodegradable light-guiding layer into an opening or cut or wound of the tissue, such cut or opening can be treated with a photo-activated dye (such as, for example, Rose Bengal or riboflavin) the emission from which, delivered towards the optical detector, is indicative of the continuing PTB within the tissue. Clinical application of an embodiment configured as a sensor include detection of cancer, inflammatory disease, hypoxic tissue, neovascularization, level of blood oxygenation, and applications in plastic and reconstructive surgery where materials embedded under grafts and flaps of the tissue can provide optical information on the ‘take” and viability of the reconstructed tissue.

It is appreciated therefore that, for the purposes of this disclosure and the appended claims, a source of light includes a laser, and LED, a broad-band source or other emitter transmitting light through the light-guiding structure towards the tissue. In addition or alternatively, a source of light includes an emitter associated with the tissue such as a fluorophore with which the tissue may be tagged, or a portion of the tissue itself that generates and transmits light through the light-guiding structure towards an optical detector outside of the tissue. In practice, light is coupled into an embodiment of the biodegradable light-guiding structure in an applicable fashion known in the art with the use of at least one optical terminal such as the terminal 210 of FIG. 2.

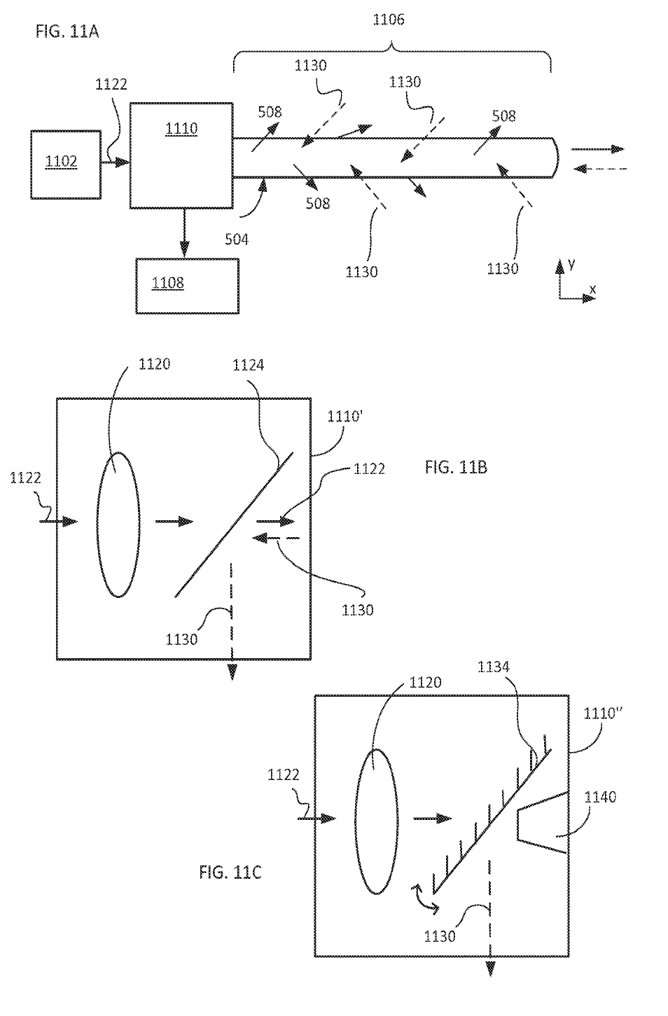

FIG. 11A shows a general schematic diagram illustrating optical communication between (i) a source of light 1102 such as a laser, or an LED, or a broad-band optical source , (ii) an embodiment of the biodegradable light-guiding layer (such as, for example, the layer 200 of FIG. 2 or 400 of FIG. 4 or 900 or 950 of FIG. 9), represented by a FO-element 1106, and (iii) and optional optical detector 1108 with the use of an optical terminal 1110. FIGS. 11B and 11C show two examples 1110′ and 1110″ of implementation of the optical terminal 1110. The embodiment 1110′ contains an optical coupling element 1120 such as a lens that facilitates the coupling of light 1122 from the source of light 1102 into the light-guiding layer 1106 through a beamsplitter 1124 (for delivery inside the tissue, not shown). The embodiment 1110″ includes, instead, a tunable diffractive optical element 1134 such as a rotatable diffraction grating and an optical taper 1140 at the input of the light-guiding layer 1106. The optical detector 1108 is adapted to register light that has been received by the layer 1106 from inside the tissue, as shown schematically by dashed arrows 1130, and that has been guided by the layer 1106 and reflected by the beamsplitter 1124. In other related embodiments, an optical terminal may include an appropriately prepared facet of the light-guiding layer 1106 or, additionally or alternatively, an optionally modified surface of the light-guiding layer 1106 through which the in-coupling of light to the layer 1106 can be effectuated.

### EXAMPLES OF USE

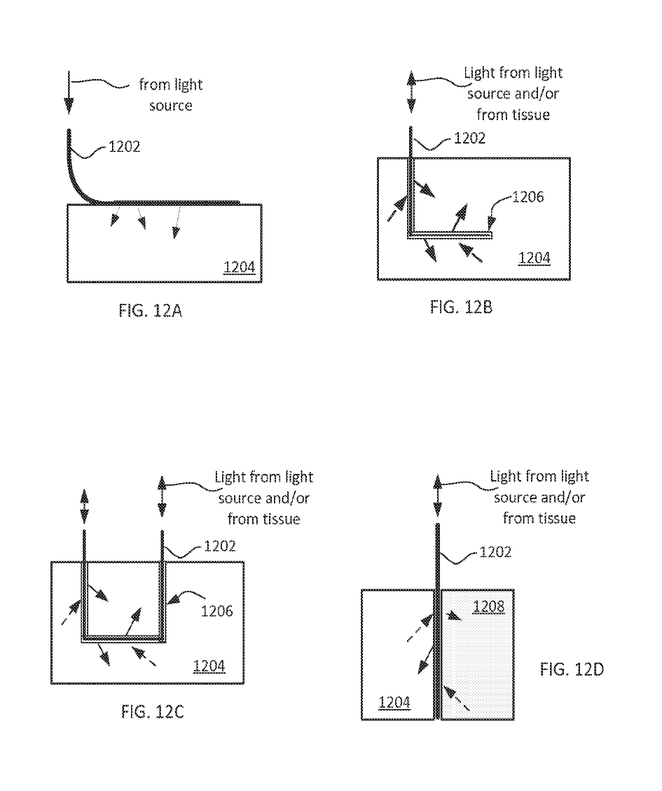

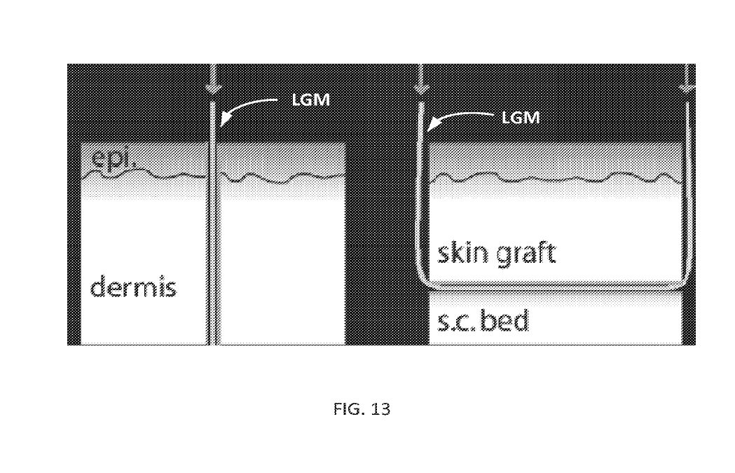

Discussion of use of the above-described biodegradable light-guiding structures are further provided in reference to FIGS. 12 through 13. Examples of practical applications supported by the embodiments of the invention include light-assisted PTB: (a) irradiation of in-depth wound facilitating wound closure, (b) horizontal tissue bonding such as skin grafting, for example, and (c) irradiation of the tissue surface. Generally, delivery of light deeper in tissue can be applied to a wide variety of medical uses. These include photodynamic therapy at depth and optical sensing of physiological tissue status, such as viability, perfusion, infection and sterilization, pH, and the like. Biodegradable light-guiding structures having variable material composition chosen to affect the resorption rate of the mesh in the tissue for different medical applications are also considered to be within the scope of the invention. For acute light delivery applications, for example, the materials, such as 50/50 PLGA, can be chosen to have high degradation and resorption rates, whereas for longer processes such as wound healing or sensing of tissue status the resorption rate could be much slower.

The following provides, without limitation, several example of practical uses of the disclosed embodiments: Low-level light therapy (LLLT) using red or near-IR light for bio-stimulation and wound healing purposes for improving recovery following stroke. For example, the light-guiding layer can be installed at the wound bed (for example, during tissue graft placement), such that in the first days following the procedure the cells in the wound bed are continually stimulated to increase rate of wound healing.

Inhibition of contractile scarring: An implanted biocompatible light delivery system used with photoactive agents to crosslink extracellular matrix proteins and to reduce the extent of contracture following major plastic surgery procedures that involve skin grafting.

Tissue surface passification against adhesion formation: In many surgical procedures, particularly abdominal, gynecological and orthopedic surgeries, scarring can occur after tissue repair in the form of adhesions between organs or tissues, causing major complications. A light conducting material can be placed over or into a wound such as the surgical wound within the body to inactivate abnormal scarring processes that form adhesions via photodynamic, photo-thermal or photo-crosslinking processes. For example, an LGM can be cooperated with a wound implant or a wound-covering element for passification of a surface towards inflammation, adhesions, capsule formation, bio-film formation, or fibrosis.

Internal deep wound closure: A light-guiding embodiment of the invention can be placed in surgical incisions or traumatic lacerations and illuminated in the presence of a photo-initiator that has been applied to the tissue surfaces to seal the tissues together across the entire interface of the laceration/incision. This is particularly applicable to deep incisions in tissues or lacerations in solid tissues such as kidney, liver, skin, muscle, connective tissue, larynx, heart and the like.

Cardiac applications: The light-guiding layer can be shaped in a tubular form to provide luminal support and homogeneous light delivery to endoluminal tissues, in order to effect biological responses to irradiating light. Non-limiting examples of useful embodiments include biocompatible and biodegradable stents for cardiac application including vulnerable plaque stabilization and photodynamic therapy of cardiac diseases. Another use of discussed embodiment includes light-activated release, into the tissue, of a vasodilator such as nitric oxide, for example, from molecules with which the vasodilator may be bound (such as molecules of glutathione) to provide local vasorelaxing effect at the side of aneurysm in order to prevent stroke following aneurysm. Tubularly-shaped and, in particular, cylindrically-shaped light-guiding embodiments can also be deployed intralumenally for light-activated surgical repair or treatment of disease in tissues such as esophagus, larynx, small and large intestine. Targeted diseases include cancer, inflammatory bowel disease, and Barrett's oesophagus, to name just a few.

Surgery: Natural orifice transluminal endoscopic surgery is a recently developed, minimally-invasive surgical procedure for intra-abdominal surgery where surgical access to the region of interest is gained from the gastrointestinal (GI) tract rather than externally through the abdomen. In one approach, access is effectuated through the stomach with a gastric flap rather than a straight puncture, to limit the possibility of the leakage of GI contents into the abdomen following the surgery. Insertion of a light delivery mesh into the flap with photo-initiator provides a method for a full seal across the entire flap.

Large internal surface treatment: Large LGMs can be delivered into the tissue through catheters or endoscopes, e.g. laparoscopy, in an “unrolling” fashion, for example, for internal deployment for photo-treatment of large surface or disseminated disease, such as in bladder, lung or intraperitoneal disease. Various photodynamic treatments could be effectively performed in this manner.

In an embodiment of FIG. 12A, the flexible or malleable biodegradable side-surface-emitting light-guiding layer 1202 (such as a WG mesh or LGM of FIG. 2 or 3, for example or a slab waveguiding layer of FIGS. 9A or 9B) is shown to be cooperated with (for example, brought in contact or affixed to) a surface of the tissue 1204 to deliver EM radiation to a wide area of the skin-layer of the tissue 1204. FIGS. 12B and 12C depict a light-guiding layer 1202 that has been inserted (implanted, embedded) into an existing in-depth wound, an incision, or a passage 1206 in the tissue 1204. FIG. 12D shows the layer 1202 sandwiched between two tissues 1204 and 1208 to facilitate tissue bonding. In further reference to FIGS. 12A through 12D, the solid arrows represent light that has been guided from the light source into the tissue via the light-guiding layer 1202, while the dashed arrows represent light coupled into the light-guiding layer 1202 from the tissue. FIG. 13 provides an additional illustration to the concept of deep embedding of an embodiment of the light-guiding layer into the biological tissue of choice.

### Demonstration

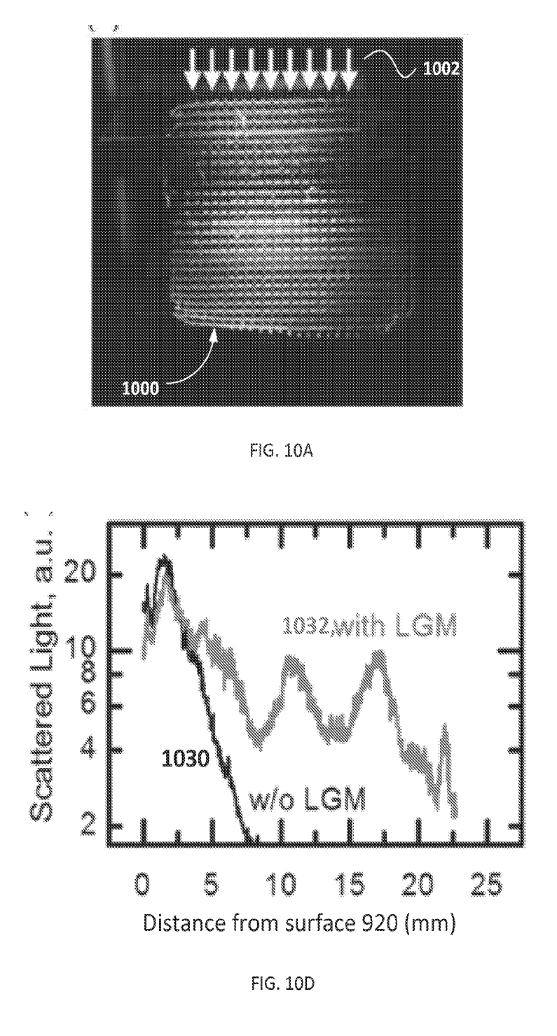

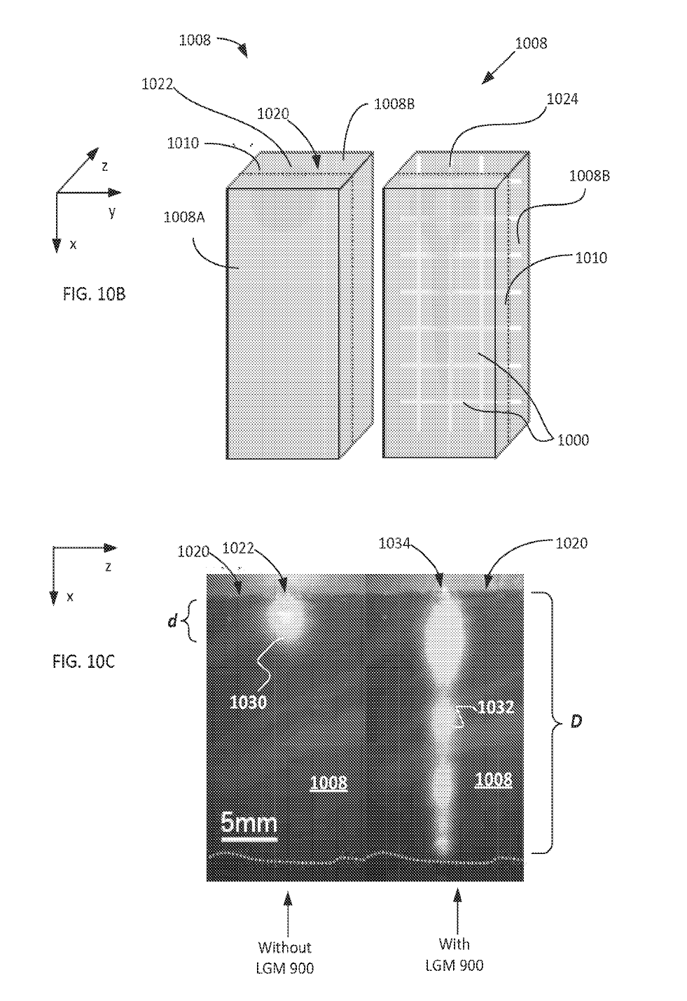

Examples illustrating the use of the embodiments are further discussed in reference to FIGS. 10A, 10B, 10C, and 10D. In particular, FIG. 10A is an image of a WG-mesh (light-guiding mesh, or LGM) 1000 fabricated from PEGDA with photolithography. The interspacing of the mesh is about 0.5 mm, and the diameters of the vertical and horizontal paths are about 0.2 mm. Arrows 1002 indicate that green (for example, 532 nm) laser light was coupled into the WG-mesh through input optical terminals distributed along the top of the WH-mesh. As shown, the LGM 1000 emits guided light across the area of the LGM primarily by scattering. The placement of the LGM 1000 into the biological tissue 1008, along a cut/incision (indicated with the dashed line 1010) that defines two portions 1008A, 1008B of the tissue 1008, is illustrated in FIGS. 10B and 10C, in perspective and cross-sectional views, respectively.

FIGS. 10B and 10C provide comparison between the depth of penetration, into the tissue, of light irradiating the tissue surface 1020 and that delivered into the tissue by the LGM 1000. In the example not employing the LGM 1000, the irradiating light was focused at a point 1022 on the incision line 1010, penetrated into the tissue 1008 at a subcutaneous depth d on the order of a couple of millimeters, and was detected by monitoring the scattered light 1030 through the xy-surface of the tissue 1008 with a camera-based imaging system. In contradistinction, the irradiation 1032 of the tissue 1008 with light coupled into the LGM 1000 at an input terminal 1034 was ensured at depths up to D>d. FIG. 10D shows intensity profiles 1030, 1032 of scattered light representing the efficiency of irradiation of tissue with light at 532 nm as a function of tissue depth and demonstrating that penetration depth ensured by the LGM 1000 is several times that achieved with direct illumination of the tissue from the surface.

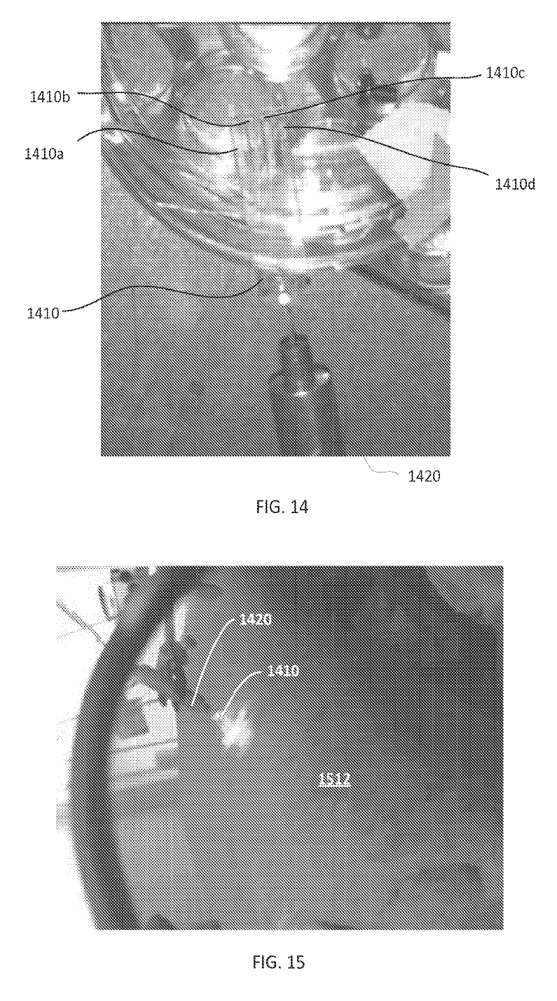

FIG. 14 shows a comb-like PLLA waveguide structure 1410 fabricated with a thickness of about 0.5 mm. The surface of the waveguide 1410 was dip-coated with collagen fibers to promote bonding between the polymeric material of the waveguide structure 1410 and the tissue, in addition to photochemical crosslinking between tissues through the spacing between the waveguide combs (four waveguide “fingers” 1410a, 1410b, 1410c, and 1410d in this example). As shown in FIG. 14, the operability of the waveguide 1410 was confirmed by coupling the light (red portion of the optical spectrum) from a laser source 1420 (the bottom cylinder in FIG. 14).

FIG. 15 provides an illustration to the tissue bonding procedure performed on a pig (biological tissue 1512) with the use of an embodiment of FIG. 14. An incision of about 2 cm in length and about 1 cm in depth was made on the skin, Rose Bengal was applied, and the finger-portion of the waveguide structure 1410 of FIG. 14 was inserted into the so formed cut. The PTB was performed using laser light in a green portion of the spectrum coupled into the waveguide such that an output power at an end of the polymer waveguide 1410 was about 1 W. After irradiation of the tissue for about 40 min, a significant photo bleaching of Rose Bengal near the waveguide fingers 1410a through 1410d was confirmed and the bonding strength of approximately 10 kPa was measured. Such bonding strength is comparable to the maximum strength of PTB-induced skin bonding. This experimental result provided, therefore, an empirical proof of feasibility and efficiency of waveguide-assisted PTB and, more generally, showed the practicality of waveguide-assisted activation of molecules in deep tissue.

In accordance with specific embodiments described with reference to FIGS. 2 through 15, a system and method are provided for supporting (i) a process of deep tissue irradiation and for (ii) extracting, from the depths of the tissue, light the spectrum of which is informative of the status of the tissue with the use of a biodegradable optical waveguide component. Modifications to, and variations of, the illustrated embodiments may be made without departing from the inventive concepts disclosed herein. Furthermore, disclosed aspects, or portions of these aspects, may be combined in ways not listed above. Accordingly, the invention should not be viewed as being limited to the disclosed embodiment(s).

What is claimed is:

1. A light-delivery system comprising:
a biodegradable mesh of optical waveguides, said optical waveguides having respectively corresponding light-guiding surfaces and terminating facets,
said biodegradable mesh having an optical terminal structured to receive light from a light source into optical waveguides of said biodegradable mesh,
wherein at least one of said optical waveguides is structured to radiate light propagating therein through at least one of (i) a corresponding light-guiding surface when said surface is in contact with the biological tissue and (ii) a corresponding terminating facet.

2. A system according to claim 1, wherein at least one of said optical waveguides includes at least one of polyethylene glycols (PEGs), poly-L-lactic acid (PLLA), poly-dl-lactide-co-glycolide (PLGA).

3. A system according to claim 1, further comprising an opto-electronic component including at least one of a) a source of light, wherein said opto-electronic component is adapted to couple light into the optical terminal of said biodegradable mesh, and b) an optical detector adapted to receive light, guided by the at least one of the optical waveguides, through the optical terminal of said biodegradable mesh.

4. A system according to claim 2, wherein said biodegradable mesh is adapted to be disposed in a crevice of a biological tissue at a depth of at least 1 cm.

5. A system according to claim 2, wherein said biodegradable mesh is adapted to collect light through at least one of the light-guiding surfaces and deliver so collected light from the biological tissue towards an optical detector disposed in optical communication with the optical terminal.

6. A system according to claim 1, further comprising particles dispersed through the at least one of the optical waveguides, the particles being structured to cause at least one of a) scattering of light guided by said at least one of the optical waveguides and b) generation of fluorescence in response to interaction between said particles and light guided by said at least one of the optical waveguides.

7. A system according to claim 1, wherein the biodegradable mesh is configured as a flexible tube.

8. A system according to claim 1, wherein the biodegradable mesh includes at least one of a malleable mesh and a flexible mesh.

9. A light-delivery system comprising:
a light-guiding layer including:
a first portion structured to receive light from a source of light, and
a second portion adapted structured to emit light that has propagated in the light-guiding layer,
wherein the light-guiding layer includes a biodegradable material.

10. A system according to claim 9, wherein said light-guiding layer includes at least one of a light-guiding surface and a facet and is adapted to emit light through at least one of said light-guiding surface and said facet.

11. (canceled)

12. (canceled)

13. (canceled)

14. A system according to claim 12, wherein the network of optical waveguides includes a mesh of optical waveguides.

15. A system according to claim 14, further comprising a source of light adapted to couple light into an optical terminal associated with the light-guiding layer, wherein the light-guiding layer is configured to emit light towards a biological tissue, and wherein mesh openings of the mesh of optical waveguides are sized to have dimensions substantially equal to a penetration depth of emitted light into the biological tissue.

16. A system according to claim 14, wherein said mesh of optical waveguides includes irregularly shaped mesh openings.

17. (canceled)

18. A system according to claim 9, wherein the light-guiding layer forms a slab waveguide.

19. A system according to claim 9, further comprising an optical system configured to couple light from the source of light to an optical terminal associated with the light-guiding layer.

20. A system according to claim 19, further comprising an optical detector positioned to receive light that has been guided by the light-guiding layer and that has emanated from said optical terminal.

21. A system according to claim 19, further comprising particles dispersed in the light-guiding layer, said particles being adapted to affect propagation of light through the light-guiding layer.

22. A system according to claim 21, wherein the particles include at least one of particles fluoresce fluorescing in response to being irradiated with light guided by the light-guiding layer and particles structured to scatter light guided by the light-guiding layer.

23. (canceled)

24. (canceled)

25. (canceled)

26. A system according to claim 9, wherein the biodegradable material includes at least one of polyethylene glycols (PEGs), poly-L-lactic acid (PLLA), poly-dl-lactide-co-glycolide (PLGA) block copolymer, silk, collagen, and a silk collagen block copolymer.

27. A system according to claim 9, wherein the light-guiding layer includes at least one flexible tubular layer.

28-46. (canceled)
